SEQUENCE LISTING (5 sequences)

SEQ ID NO: 1 - DNA, 21 bp, Artificial
  Forward primer sequence for HIV-1, 357-gag183U (gag183U)
ctagcagtggcgcccgaacag

SEQ ID NO: 2 - DNA, 31 bp, Artificial
  Reverse primer sequence for HIV-1, 292-gag187L (gag187L)
ccatctctctccttctagcctccgctagtca

SEQ ID NO: 3 - DNA, 27 bp, Artificial
  Probe sequence for HIV-1, 354-gag187PF (gag187P)
tctctcgacgcaggactcggcttgctg

SEQ ID NO: 4 - DNA, 80 bp, Artificial
  IC molecule sequence; 352-gag187IC-C4
gtggcgcccgaacagtatcgcgtttatgcgaggtcgggtgggcgggtcgttagtttcgttttgggtgactagcggaggct

SEQ ID NO: 5 - DNA, 22 bp, Artificial
  Probe sequence for IC; 361-gag187ICP2
aggtcgggtgggcgggtcgtta

(19) United States
(12) Patent Application Publication
(10) Pub. No.: US 2012/0135397 A1
(43) Pub. Date: May 31, 2012

(54) HUMAN IMMUNODEFICIENCY VIRUS TYPE 1 (HIV-1) DETECTION METHOD AND KIT THEREFOR

(75) Inventors: Masafumi Inoue, Singapore (SG); Oon Tek NG, Singapore (SG)

(73) Assignees: TAN TOCK SENG HOSPITAL, Singapore (SG); AGENCY FOR SCIENCE, TECHNOLOGY AND RESEARCH, Singapore (SG)

(21) Appl. No.: 13/382,757

(22) PCT Filed: Jul. 7, 2010
(86) PCT No.: PCT/SG10/00257

§ 371 Date: Jan. 6, 2012

Related U.S. Application Data
(60) Provisional application No. 61/223,638, filed on Jul. 7, 2009.

(51) Int. Cl. C12Q 1/70; C12P 19/34; C07H 21/04

(52) U.S. Cl. 435/5; 536/24.33; 536/24.32; 536/23.72; 435/91.2

(57) ABSTRACT

The invention provides oligonucleotide(s) derived from the gene sequence encoding the gag region of HIV-I for simple, specific and/or sensitive test(s) for the presence of HIV-I. In particular, the present invention provides oligonucleotide(s) for test(s) for HIV-I. Kit(s) comprising the oligonucleotide(s) for use as probe(s) and/or primer(s) useful in the test(s) are also provided.

### FIELD OF THE INVENTION

The present invention relates to primer(s), probes as well as method(s) and kit(s) using such primer(s) and/or probes for the detection of the presence of human immunodeficiency virus type 1 (HIV-1).

### BACKGROUND TO THE INVENTION

HIV is one of the most serious infectious diseases in the world and is a global pandemic with the most recent World Health Organizations' report in November 2009 estimating 33.4 million infections worldwide and about 2 million deaths in 2008. The WHO has also listed “combat HIV/AIDS” as the 6th millennium development goal (http://www.who.int/mdg/en/).

This global pandemic has been met by an unprecedented effort to make available treatments, especially to low and middle-income developing countries. By December 2007, an average of 3 million people living with HIV-1 in these countries was receiving anti-retroviral therapy, only 31% of all who needed it. With the annual increase in HIV-1 cases and the slow increase in the number of people getting tested, it is expected that an estimated 7 million individuals in developing countries will be on therapy by 2010.

Early detection of HIV-1 infection is important so that treatment can begin early. HIV-1 viral detection is also the only means of diagnosing early mother-to-child transmission of virus. HIV-1 viral quantitation (i.e. viral load) is the most sensitive prognostic marker of long-term response to treatment as no surrogate marker has been able to replace the viral load for monitoring treatment response. Virologic suppression to undetectable levels is in most cases, the primary endpoint for many randomised treatment trials and the key goal in many treatment guidelines. Virologic suppression minimises the risk of development of resistance to treatment.

Since the majority of HIV-1 infected persons reside in developing countries where access to viral load monitoring is limited due to the need for extensive laboratory facilities, trained personnel and financial costs, a lot of HIV-1 infected people live with the infection without seeking treatment for they have no idea of their infection. Real-time PCR assays also require refrigeration for reagents, multiple rooms to prevent contamination and many expensive instruments for the various processes which further hamper access to viral load monitoring.

For example, the cost per assay is approximately US$150 and the costs of genotyping about US$550 per sample. The average frequency of viral load monitoring is 0.7 viral loads per patient per year, a gross underutilization of an essential treatment-monitoring tool. Some centres have developed in-house HIV quantitation assays in an effort to improve throughput and lower costs. Multiple studies have been conducted demonstrating that in-house viral load quantitation and genotyping is feasible and significantly cheaper. For example, the cost of viral load quantitation is reported to be as low as [symbol] 20 and genotyping another [symbol] 35 per run. However, one of the potential limitations in this strategy is the need for Biosafety Level 3 (BSL3) level laboratories for viral culture for internal standards.

### SUMMARY OF THE INVENTION

The present invention is defined in the appended independent claims. Some optional features of the present invention are defined in the appended dependent claims. In particular, the present invention addresses the problems above, and provides highly sensitive and specific oligonucleotides, fragments and/or derivatives thereof useful in a method of detecting and quantitating HIV-1 in patient specimens more efficiently. The primers and/or probes may be sensitive and specific in the detection of HIV-1 and provide rapid and cost-effective diagnostic and prognostic reagents for determining infection by HIV-1 and/or disease conditions associated therewith. These primers provide a means for cheap, fast and more accurate HIV-1 testing. In particular, these primers provide an affordable point-of-care HIV-1 quantitation assay which is comparable to current commercial kits with only the use of routine enhanced BSL2 facilities thereby increasing access to viral load testing with a more affordable kit.

According to a first aspect, the present invention provides an isolated oligonucleotide comprising, consisting essentially of, or consisting of at least one nucleotide sequence selected from the group consisting of: SEQ ID NO:1 to SEQ ID NO:3, fragment(s), derivative(s), mutation(s), and complementary sequence(s) thereof. The oligonucleotide may be capable of binding to and/or being amplified from HIV-1.

According to another aspect, the present invention provides at least one pair of oligonucleotides comprising at least one forward primer and at least one reverse primer, wherein the forward primer comprises, consists essentially of or consists of SEQ ID NO:1, fragment(s), derivative(s), mutation(s), or complementary sequence(s) thereof and the reverse primer comprises, consists essentially of or consists of SEQ ID NO:2, fragment(s), derivative(s), mutation(s), or complementary sequence(s) thereof.

According to another aspect, the present invention provides at least one set of oligonucleotides comprising a pair of oligonucleotides according to any aspect of the present invention and at least one probe.

According to a further aspect, the present invention provides at least one amplicon amplified from HIV-1 using at least one forward primer comprising, consisting essentially of or consisting of the nucleotide sequence of SEQ ID NO:1, fragment(s), derivative(s), mutation(s), or complementary sequence(s) thereof and at least one reverse primer comprising, consisting essentially of or consisting of the nucleotide sequence of SEQ ID NO:2 fragment(s), derivative(s), mutation(s), or complementary sequence(s) thereof.

According to one aspect, the present invention provides at least one method of detecting the presence of HIV-1 in a biological sample, the method comprising the steps of:
(a) providing at least one biological sample;
(b) contacting at least one oligonucleotide, pair of oligonucleotides or set of oligonucleotides according to any aspect of the present invention, with at least one nucleic acid in the biological sample, and/or with at least one nucleic acid extracted, purified and/or amplified from the biological sample; and
(c) detecting any binding resulting from the contacting in step (b) whereby the HIV-1 is present when binding is detected.

According to one aspect, the present invention provides at least one method of amplifying HIV-1 nucleic acid, wherein said method comprises carrying out a polymerase chain reaction using SEQ ID NO:1, fragment(s), derivative(s), mutation(s), or complementary sequence(s) thereof and SEQ ID NO:2 fragment(s), derivative(s), mutation(s), or complementary sequence(s) thereof.

According to another aspect, the present invention provides at least one kit for the detection of HIV-1, the kit comprising at least one oligonucleotide, pair of oligonucleotides or set of oligonucleotides according to any aspect of the present invention.

According to a particular aspect, there are provided highly sensitive and specific primers, fragments and/or derivatives thereof useful in a method of PCR capable of detecting HIV-1 DNA in patient specimens. This test may be used to examine the specimens from patients with HIV-1. The primers may be sensitive and specific.

Further, at least one IC molecule may be included in each reaction to monitor the PCR performance. ICs may be considered to be an important feature as inhibition rates can be as high as 3.7% in large-scale validations.

The oligonucleotides according to any aspect of the present invention may be used in an in-house assay suitable for clinical use in patient monitoring. It may be comparable to current commercially available assays in quantitating over 300 patient samples. In particular, the oligonucleotides according to any aspect of the present invention may be comparable clinical performance with recently large-scale evaluated in-house assays currently in clinical use.

The Sing-IH has been applied on a prototype portable platform. A prototype device using this real-time RT-PCR assay was presented at the 2009 International AIDS Society meeting in South Africa (Ng et al., 2009). In brief, the portable, credit-card-sized real-time device using the protocol described here was demonstrated to accurately quantify HIV-1 cDNA with an r2 value of 1.00 compared to the benchtop size Stratagene Mx 3000P real-time PCR system (Strategene, La Jolla, USA).

The Sing-IH assay may be considered to be a reliable, easy to use, affordable assay to increase access for reliable monitoring of response to therapy. In particular, it may be useful in regions where subtype B and AE predominate. The total cost of all the reagents was less than US$10 per reaction. Also, it may have a lower limit of detection ranging from 50 copies/ml to 400 copies/ml thus making it a sensitive assay for detection of HIV-1.

As will be apparent from the following description, preferred embodiments of the present invention allow for an optimal use of the primers and/or probes for the sensitive and specific the detection of HIV-1 where desired. This and other related advantages will be apparent to skilled persons from the description below.

### BRIEF DESCRIPTION OF THE FIGURES

FIG. 1 is a table of a list of mutations in the reverse transcriptase gene of HIV-1 associated with resistance to reverse transcriptase inhibitors (Johnson et al., 2009).

FIG. 2 A-C is a table of a list of mutations in the (A) protease gene of HIV-1 associated with resistance to protease inhibitors; (B) envelope gene of HIV-1 associated with resistance to entry inhibitors; (C) integrase gene of HIV-1 associated with resistance to integrase inhibitors (Johnson et al., 2009).

FIG. 3 is a HIV-1 standard curve showing the linear relationship between the cycle number (Ct) and the serially diluted plasmid standards (10¹-10⁸ copies) using the in-house assay of the present invention.

FIG. 4 is a graph of Probit regression showing the limit of detection for the in-house assay (dotted lines show the 95% confidence interval) from Example 1

FIGS. 5A and B are Bland-Altman plots of valid quantitative results for clinical plasma samples carried out in Example 1. Graph A shows a comparison of the in-house assay (IH) and COBAS TaqMan HIV-1 test (CTM) (n=118). Graph B shows a comparison of the in-house assay (IH) and Abbott Real time HIV-1 test (ART) (n=97).

FIGS. 6 A and B are histograms of the log₁₀ viral load values for the in-house assay, CTM and ART assays carried out in Example 1. Graph A shows the comparison between the in-house and CTM (n=118). Graph B shows the comparison between the in-house and ART (n=97).

FIG. 7 is a graph of Probit regression showing the limit of detection for the in-house assay (dotted lines show the 95% confidence interval) from Example 2.

FIGS. 8A and B are Bland-Altman plots of valid quantitative results for clinical plasma samples carried out in Example 2. Graph A shows comparison of the in-house assay (i.e. Sing-IH) with the COBAS TaqMan HIV-1 test (n=119). Graph B shows comparison of the in-house assay (i.e. Sing-IH) with Abbott Real time HIV-1 test (n=108).

FIGS. 9A and B are histograms of the log₁₀, viral load values for the in-house, COBAS TaqMan HIV-1 and Abbott Real-Time HIV-1 assays carried out in Example 2. Graph A shows the comparison between Sing-IH and COBAS TaqMan HIV-1 (n=119). Graph B shows the comparison between Sing-IH and Abbott Real-Time HIV-1 (n=108).

### DETAILED DESCRIPTION OF THE PREFERRED EMBODIMENTS

Bibliographic references mentioned in the present specification are for convenience listed in the form of a list of references and added at the end of the examples. The whole content of such bibliographic references is herein incorporated by reference.

### Definitions

The term “biological sample” is herein defined as a sample of any tissue and/or fluid from at least one animal and/or plant. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Environmental samples include environmental material such as surface matter, soil, water, air and industrial samples, as well as samples obtained from food and dairy processing instruments, apparatus, equipment, utensils, disposable and non-disposable items. These examples are not to be construed as limiting the sample types applicable to the methods disclosed herein. In particular, a biological sample may be of any tissue and/or fluid from at least a human being.

The term “complementary” is used herein in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” The degree of complementarity between nucleic acid strands has significant effects on the efficiency and strength of hybridization between nucleic acid strands. This is of particular importance in amplification reactions, as well as detection methods that depend upon binding between nucleic acids. In particular, the “complementary sequence” refers to an oligonucleotide which, when aligned with the nucleic acid sequence such that the 5′ end of one sequence is paired with the 3′ end of the other, is in “anti-parallel association.” Certain bases not commonly found in natural nucleic acids may be included in the nucleic acids disclosed herein and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. Where a first oligonucleotide is complementary to a region of a target nucleic acid and a second oligonucleotide has complementary to the same region (or a portion of this region) a “region of overlap” exists along the target nucleic acid. The degree of overlap may vary depending upon the extent of the complementarity.

The term “comprising” is herein defined as “including principally, but not necessarily solely”. Furthermore, the term “comprising” will be automatically read by the person skilled in the art as including “consisting of”. The variations of the word “comprising”, such as “comprise” and “comprises”, have correspondingly varied meanings.

The term “derivative,” is herein defined as the chemical modification of the oligonucleotides of the present invention, or of a polynucleotide sequence complementary to the oligonucleotides. Chemical modifications of a polynucleotide sequence can include, for example, replacement of hydrogen by an alkyl, acyl, or amino group.

The term “fragment” is herein defined as an incomplete or isolated portion of the full sequence of an oligonucleotide which comprises the active/binding site(s) that confers the sequence with the characteristics and function of the oligonucleotide. In particular, it may be shorter by at least one nucleotide or amino acid. More in particular, the fragment comprises the binding site(s) that enable the oligonucleotide to bind HIV-1. In particular, the fragment of the forward primer may comprise at least 10, 12, 15, 18 or 19 consecutive nucleotides of SEQ ID NO:1 and/or the reverse primer may comprise at least 10, 12, 15, 18, 19, 20, 22, or 24 consecutive nucleotides of SEQ ID NO:2. More in particular, the fragment of the primer may be at least 15 nucleotides in length.

The term “internal control (IC) molecule” is herein defined as the in vitro transcribed oligonucleotide molecule which is co-amplified by the same primer set for HIV-1 used in the method of the present invention. In particular, the IC may be mixed in the reaction mixture to monitor the performance of PCR to avoid false negative results. The probe to detect this IC molecule may be specific to the interior part of this molecule. This interior part may be artificially designed and may not occur in nature.

The term “mutation” is herein defined as a change in the nucleic acid sequence of a length of nucleotides. A person skilled in the art will appreciate that small mutations, particularly point mutations of substitution, deletion and/or insertion has little impact on the stretch of nucleotides, particularly when the nucleic acids are used as probes. Accordingly, the oligonucleotide(s) according to the present invention encompasses mutation(s) of substitution(s), deletion(s) and/or insertion(s) of at least one nucleotide. Further, the oligonucleotide(s) and derivative(s) thereof according to the present invention may also function as probe(s) and hence, any oligonucleotide(s) referred to herein also encompasses their mutations and derivatives.

The term “nucleic acid in the biological sample” refers to any sample that contains nucleic acids (RNA or DNA). In particular, sources of nucleic acids are biological samples including, but not limited to blood, saliva, cerebral spinal fluid, pleural fluid, milk, lymph, sputum and semen.

According to one aspect, the present invention provides at least one isolated oligonucleotide comprising or consisting of at least one nucleotide sequence of SEQ ID NO:1 or SEQ ID NO:2, fragment(s), derivative(s), mutation(s) or complementary sequence(s) thereof. The oligonucleotide may be capable of binding to and/or being amplified from HIV-1. The HIV-1 may be of group M, further divided into subtype A-K, or of group N, O or P.

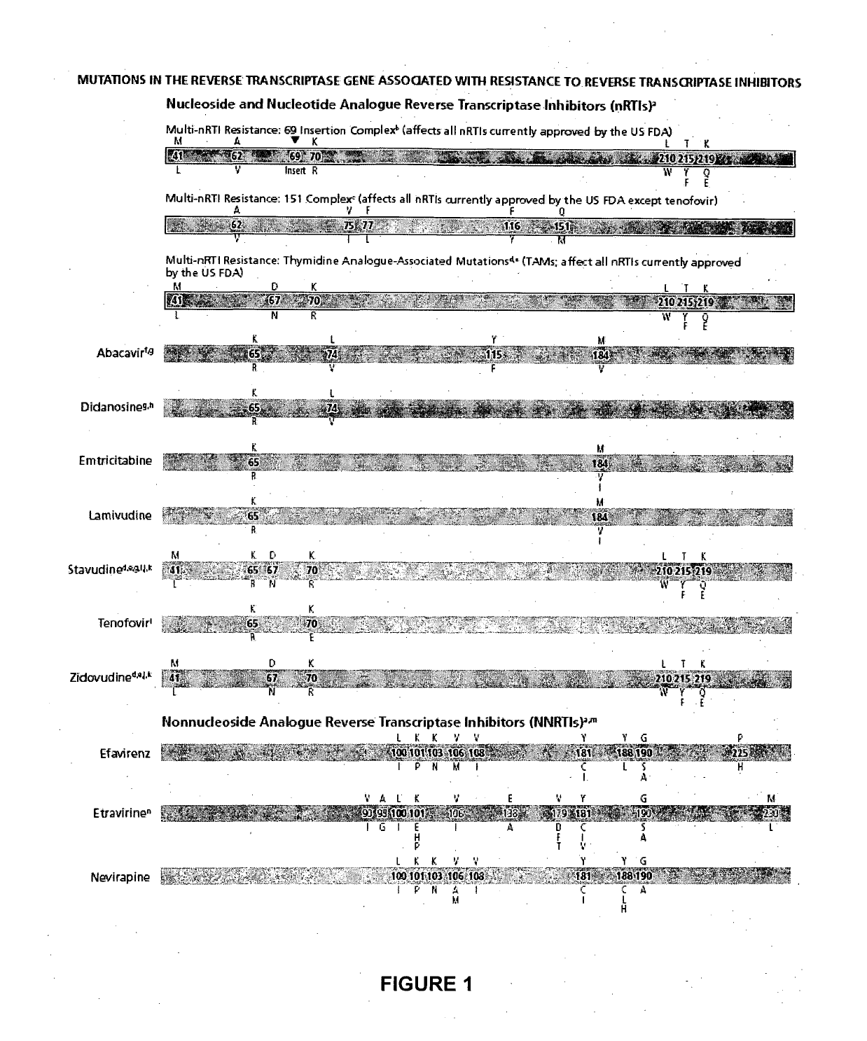

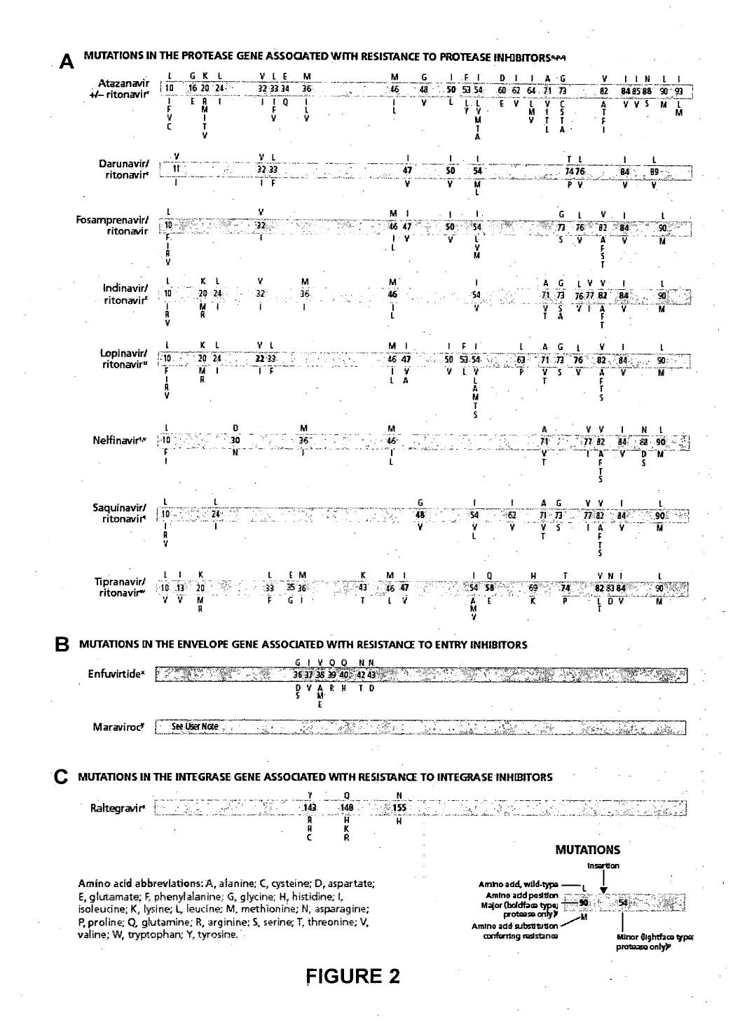

The HIV-1 genotype may comprise a drug-resistant strain. The drug resistance may be found in the reverse transcriptase, envelope, protease and/or integrase gene of HIV-1. In particular, the drug resistant strain of HIV-1 may be resistant to one or more drugs selected from the group consisting of: abacavir, atazanavir, darunavir, delavirdine, didanosine, efavirenz, emtricitabine, enfuvirtide, etravirine, fosamprenavir, indinavir, lamivudine, lopinavir, nelfinavir, nevirapine, raltegravir, ritonavir, saquinavir, stavudine, tenofovir, tiprannavir, and zidovudine. A non-limiting list of mutations of different genes of HIV-1 is found in FIGS. 1 and 2. The drug resistant strain of HIV-1 may be a multi-drug resistant strain which may be resistant to a plurality of drugs selected from the group consisting of: atazanavir, darunavir, fosamprenavir, indinavir, lopinavir, nelfinavir, Saquinavir, tipranavir and ritonavir. The oligonucleotide according to any aspect of the present invention may bind to HIV-1 with one or more of these mutations

The oligonucleotide sequence may be between 13 and 35 linked nucleotides in length and may comprise at least 70% sequence identity to SEQ ID NO:1, SEQ ID NO:2 or SEQ ID NO:3. A skilled person will appreciate that a given primer need not hybridize with 100% complementarity in order to effectively prime the synthesis of a complementary nucleic acid strand in an amplification reaction. A primer may hybridize over one or more segments such that intervening or adjacent segments are not involved in the hybridization event, (e.g., for example, a loop structure or a hairpin structure). In particular, the sequence of the oligonucleotide may have 80%, 85%, 90%, 95% or 98% sequence identity to SEQ ID NO:1, SEQ ID NO:2 or SEQ ID NO:3.

An extent of variation of 70% to 100%, or any range there within, of the sequence identity is possible relative to the specific primer sequences disclosed. Determination of sequence identity is described in the following example: a primer 20 nucleotides in length which is identical to another 20 nucleotides in length primer having two non-identical residues has 18 of 20 identical residues (18/20=0.9 or 90% sequence identity). In another example, a primer 15 nucleotides in length having all residues identical to a 15 nucleotides segment of primer 20 nucleobases in length would have 15/20=0.75 or 75% sequence identity with the 20 nucleotides primer.

Percent homology, sequence identity or complementarity, can be determined by, for example, the Gap program (Wisconsin Sequence Analysis Package, Version 8 for UNIX, Genetics Computer Group, University Research Park, Madison Wis.), using default settings, which uses the algorithm of Smith and Waterman known in the art. A skilled person is able to calculate percent sequence identity or percent sequence homology and able to determine, without undue experimentation, the effects of variation of primer sequence identity on the function of the primer in its role in priming synthesis of a complementary strand of nucleic acid for production of an amplification product.

According to another aspect, the present invention provides at least one pair of oligonucleotides comprising at least one forward primer and at least one reverse primer wherein the forward primer comprises, consists essentially of or consists of SEQ ID NO:1, fragment(s), derivative(s), mutation(s), and complementary sequence(s) thereof and the reverse primer comprises, consists essentially of or consists of SEQ ID NO:2, fragment(s), derivative(s), mutation(s), and complementary sequence(s) thereof.

According to another aspect, the present invention provides at least one set of oligonucleotides comprising a pair of oligonucleotides according to any aspect of the present invention and at least one probe. The probe may comprise, consists essentially of or consists of SEQ ID NO:3.

The probe may be labeled with a fluorescent dye at 5′ and 3′ ends thereof. Examples of the 5′-labeled fluorescent dye may include, but are not limited to, 6-carboxyfluorescein (FAM), hexachloro-6-carboxyfluorescein (HEX), tetrachloro-o-carboxyfluorescein, and Cyanine-5 (Cy5). Examples of the 3′-labeled fluorescent dye may include, but are not limited to, 5-carboxytetramethylrhodamine (TAMRA) and black hole quencher-1,2,3 (BHQ-1,2,3).

The oligonucleotide according to any aspect of the present invention may be used in a method for the detection of HIV-1 from either a clinical or a culture sample, wherein the clinical samples may be selected from but are not limited to blood, serum, plasma, sputum, urine, stool, skin, cerebrospinal fluid, saliva, gastric secretions, semen, seminal fluid, breastmilk, tears, oropharyngeal swabs, nasopharyngeal swabs, throat swabs, nasal aspirates, nasal wash, fluids collected from the ear, eye, mouth, respiratory airways, spinal tissue or fluid, cerebral fluid, trigeminal ganglion sample, a sacral ganglion sample, adipose tissue, lymphoid tissue, placental tissue, upper reproductive tract and the like.

According to a further aspect, the present invention provides at least one amplicon amplified from HIV-1 using at least one forward primer comprising, consisting essentially of or consisting of the nucleotide sequence of SEQ ID. NO:1 and at least one reverse primer comprising, consisting essentially of or consisting of the nucleotide sequence of SEQ ID NO:2. A probe according to any aspect of the present invention may be capable of binding to the amplicon. In particular, the probe comprises, consists essentially of or consists of the nucleotide sequence of SEQ ID NO:3.

According to one aspect, the present invention provides at least one method of detecting the presence of HIV-1 in a biological sample, the method comprising the steps of:
(a) providing at least one biological sample;
(b) contacting at least one oligonucleotide, pair of oligonucleotides or set of oligonucleotides according to any aspect of the present invention, with at least one nucleic acid in the biological sample, and/or with at least one nucleic acid extracted, purified and/or amplified from the biological sample; and
(c) detecting any binding resulting from the contacting in step (b) whereby HIV-1 is present when binding is detected.

The method may be used for determining the identity and quantity of HIV-1 in a sample comprising contacting the sample with a pair of primers according to any aspect of the present invention and a known quantity of a calibration polynucleotide comprising a calibration sequence, concurrently amplifying nucleic acid from the HIV-1 in the sample with the pair of primers and amplifying nucleic acid from the calibration polynucleotide in the sample with the pair of primers to obtain a first amplification product comprising a HIV-1 identifying amplicon and a second amplification product comprising a calibration amplicon, obtaining molecular mass and abundance data for the HIV-1 identifying amplicon and for the calibration amplicon wherein the 5′ and 3′ ends of the HIV-1 identifying amplicon and the calibration amplicon are the sequences of the pair of primers or complements thereof, and distinguishing the HIV-1 identifying amplicon from the calibration amplicon based on their respective molecular masses, wherein the molecular mass of the HIV-1 identifying amplicon indicates the identity of the HIV-1, and comparison of HIV-1 identifying amplicon abundance data and calibration amplicon abundance data indicates the quantity of HIV-1 in the sample.

According to one aspect, the present invention provides at least one method of amplifying HIV-1 nucleic acid, wherein said method comprises carrying out a polymerase chain reaction using SEQ ID NO:1 and SEQ ID NO:2.

The method according to any aspect of the present invention may further comprise a step of mixing an internal molecule (IC) and a probe specific to the IC with the biological sample. The IC may comprise, consists essentially of or consists of the nucleotide sequence of SEQ ID NO:4. The IC probe may comprise, consists essentially of or consists of the nucleotide sequence of SEQ ID NO:5. The use of the IC may improve the efficiency of the HIV-1 diagnosis increasing the accuracy of results.

The method according to any aspect of the present invention may be used in PCR amplification for specific diagnosis of wherein the non-limiting HIV-1 associated condition or disease is AIDS, Kaposi's sarcoma, Non-Hodgkins lymphoma, Pneumocystis cannii pneumonia, Pneumocystis jiroveci pneumonia, Candida esophagitis, Candida albicans infection, Pseudomonas aeruginosa infection, Staphylococcus aureus infection, Streptococcus pyogenes infection, Acmetobacter baumanni infection, Toxoplasma gondii infection, Toxoplasma encephalitis, Aspergillus infection, cryptosporidiosis, microspondiosis, Cryptococcus neoformans infection, mycobacterium avium complex disseminated infection, Epstein-Barr virus infection, cytomegalovirus retinitis, progressive multifocal leukoencephalopathy from JC virus infection, HIV-associated dementia, central nervous system (CNS) malignancies, oral candidiasis, aseptic meningoencephalitis, disorders of the digestive tract, endocrine dysfunction, metabolic disorders, wasting syndrome, anemia, neutropenia, rheumatological syndromes, cervical cancer, anal cancer, rectal cancer, Burkitt's lymphoma, penicilliosis, tuberculosis, herpes virus 8 infection, herpes virus simplex 1 infection, human papillomavirus infection, cytomegalovirus infection, mycobacterial infection, rotavirus infection, adenovirus infection, astrovirus infection, esophagitis, chronic diarrhea due to Salmonella, Shigella, Listeria, or Campylobacter, or nephropathy and the like.

According to another aspect, the present invention provides at least one kit for the detection of HIV-1, the kit comprising at least one oligonucleotide, pair of oligonucleotides or set of oligonucleotides according to any aspect of the present invention.

The oligonucleotides according to any aspect of the present invention may be used in an in-house assay using BSL2 facilities in the absence of viral culture facilities and the results may be comparable to reference commercial assays. These in-house assays may also be more affordable, reliable and easy to use, thus may be used to increase the access to reliable monitoring of response to therapy in HIV-1 patients.

It is submitted that the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

An evaluation of an in-house assay (i.e. Sing-IH) using the Stratagene Mx3000 QPCR system. Clinical evaluation of the assay was performed comparing the results of the in-house assay with: (i) results from the Abbott RealTime HIV-1 assay (Abbott Molecular Inc, Des Plaines, USA) in 178 patient samples; (ii) results from the COBAS TaqMan HIV-1 test (Roche Molecular Systems, Inc., Branchburg, N.J., USA) in 151 patient samples. Detection of HIV-1 subtypes was evaluated against a genotype panel obtained from the National Institute of Biological Standards and Controls (NIBSC).

### Methods and Materials

### External Standard for Real Time Quantitation

External standard (ES) for quantitation of viral RNA copies were synthesized from RT-PCR amplification of HIV-1 positive patient sample (subtype AE) using primers gag183U (SEQ ID NO:1) and gag187L (SEQ ID NO:2), targeting the gag region. Amplicons generated by the gag183U/187L primers were verified by conventional gel electrophoresis and purified from 3% agarose gel using QIAquick Gel Extraction Kit (catalog no.: 28106, Qiagen, Germany). The Gel-purified product was further amplified using HotStarTaq Master mix kit (catalog no.: 203443, Qiagen, Germany) using primers gag183U/187L (SEQ ID NO:1 and SEQ ID NO:2 respectively). Amplicons were purified using QIAquick PCR Purification Kit (Qiagen, Germany) before cloning it with TOPO® TA Cloning® Kit for Sequencing (pCR® 4 TOPO® Vector) (catalog no.: 45-0030, Invitrogen, Carlsbad), following manufacturer's protocol. The resultant plasmids were purified, and serially diluted in 0.1 ng/uL of ssDNA and stored in −30° C. for future assay.

The insert was confirmed by sequencing performed on an Applied Biosystem 3730xl DNA analyzer using Big Dye® Terminator (version 3.1) Cycle-Sequencing Kit (Applied Biosystem, USA). The ES was calibrated against imported WHO HIV-1 RNA 2^{nd} International Standard (National Institute of Biological Standards and Control, Potters Bar, United Kingdom).

### Primer/Oligonucleotide Design

In-house primers and probes were designed using alignment data referenced from the Los Alamos National Laboratory database in 2008 (Los Alamos National Laboratory, Los Alamos, N.M). The gag gene was chosen, as it is relatively well conserved in HIV-1 virus. In particular, the sets of primers and probes used for amplification and detection were derived from the gene sequence encoding the gag region of HIV-1 genotype B and AE. Those for genotype B were chosen from the corresponding HXB2 reference strain (GenBank accession no. K03455). Those for genotype AE were chosen from the corresponding USA strain (GenBank accession no. AF259955).

The sequences of the in-house primers and probes were compared with all genotypes/groups listed in the “HIV Sequence Compendium 2008” (31 Dec. 2008) and were found to possibly bind with all groups including B and AE except O and CPZ. Genotype B and AE are predominant in Singapore. The detection oligonucleotide probe had a reporter fluorescein dye (FAM, 6-carboxyfluorescein) attached to the 5′ end and BHQ-1 quencher linked to the 3′ end.

The primers and probes used were:

Forward primer sequence for HIV-1, 357-gag183U (gag183U):

(SEQ ID NO: 1)
(5′-3′)CTAGCAGTGGCGCCCGAACAG

Reverse primer sequence for HIV-1, 292-gag187L (gag187L):

(SEQ ID NO: 2)
(5′-3′)CCATCTCTCTCCTTCTAGCCTCCGCTAGTCA

Probe sequence for HIV-1, 354-gag187 PF (gag187P):

(SEQ ID NO: 3)
(FAM) 5′-TCTCTCGACGCAGGACTCGGCTTGCTG-3′(BHQ1)

Internal control (IC)

A random sequence of a competitive internal control (IC) was designed to incorporate a unique probe-binding site different from the binding site of the HIV-1 target molecule. This competitive IC molecule was used to represent an in vitro transcribed oligonucleotide molecule, which was amplified with the primers gag187U/187L (SEQ ID NO:1 and SEQ ID NO:2 respectively). Amplification was carried out using FINNZYMES Phire™ Hot Star Taq DNA polymerase (catalog no.: F-120S, Finnzymes, Finland). This chimerical single strand DNA retaining hybridization sites at both ends of the molecule for specific primers gag183U and gag187L for HIV-1 was co-amplified to eliminate false-negative results. The IC molecule was diluted using 1 ng/μL tRNA to 100 copies/4 which was added to each of the reaction well of the real time PCR assay (i.e. one hundred copies of IC were added to each reaction of the real-time RT-PCR assay) to serve as a check for PCR inhibition. The calibration experiments showed no interference with target HIV-1 detection.

IC molecule sequence; 352-gag1871C-C4: (5′-3′)

(SEQ ID NO: 4)
5′GTGGCGCCCGAACAGTATCGCGTTTATGCGAGGTCGGGTGGGCGG
GTCGTTAGTTTCGTTTTGGGTGACTAGCGGAGGCT 3′

Probe sequence for IC; 361-gag1871CP2:

(SEQ ID NO: 5)
(Texas) 5′-AGGTCGGGTGGGCGGGTCGTTA-3′(BHQ2).

### WHO NCBIS International HIV-1 Standards

WHO international standard reagent (National institute of Biological Standards and Council (NCBIS)): HIV-1 RNA 2nd International Standard (NIBSC code: 97/650), HIV-1 RNA Working reagent 2 (NIBSC code: 99/636) and HIV-1 RNA Working Reagent 3 (NIBSC code: 05/158), containing 5.56 log₁₀ 1 U/ml, 4.56 log₁₀ IU/mL, 2.56 log₁₀ IU/mL HIV-1 respectively, were used as reference plasma to calibrate the external HIV-1 standard plasmids. The external HIV-1 was calculated to give a conversion factor of 1 IU=0.55 copies. HIV-1 RNA genotypes, 1st International Reference panel (NCBIS code: 01/466) containing HIV-1 genotypes Group M (A, B, C, D, AE, F, G, AA-GH), group N and Group O was used to determine the specificity of the in-house assay.

### Commercially Available HIV-1 Viral Load Kits Used

For Abbott Real time HIV-1 (ART) (Abbott molecular Inc.), plasma RNA was extracted using Abbott m1000sp automated sample preparation system, which used magnetic particle technology to capture the nuclei acid extracted from 1 mL of plasma. Amplification was performed on an Abbott m2000rt instrument, which targeted the integrase region of HIV-1.

COBAS Ampliprep/COBAS TaqMas HIV-1 test (Roche Molecular Systems, USA) combined automated isolation of nuclei acid on the COBAS Ampliprep instrument, using generic silica based capture technique with the automated amplification, targeting the gag-region of HIV-1 and detection on the COBAS® Taqman® Analyzer using AMPLILINK 3.1.1 software. The dynamic range of beth assays is 40-10⁷ copies/m L.

### HIV-Patient Samples

Patient samples were obtained from consented HIV-1 infected patients (both treated and newly-diagnosed) of the Communicable Disease Centre, Singapore National HIV Reference Centre. Whole blood samples were centrifuged at 3000 g for 15 minutes within 6 hours of collection and plasma obtained was stored at −80° C. until use (i.e. batch processed with the commercial and in-house assays).

### HIV-1 Viral Load was Monitored by Abbott Real Time HIV-1 Test (Art) and COBAS

TaqMas HIV-1 test (CTM) carried out by the Microbiology Laboratory of Tan Tock Seng Hospital (Singapore), and the Molecular Diagnostic Center of National University Hospital (Singapore), respectively, where they were tested based on manufacturers' protocols.

### RNA Extraction

Viral RNA was extracted from 1 mL of frozen (−80° C.) plasma ultra-centrifuged at 24,000×g for 60 minutes at 4° C. prior to extraction. 800 μL of supernatant was discarded and the remaining 2004 of plasma containing the viral pellet was used for RNA extraction using QIAamp Viral RNA mini kit (catalog no.: 52906, Qiagen, GmbH, Hilden, Germany), following manufacturer's protocol. Purified RNA was eluted in 504 of AVE buffer and stored at −80° C.

### Real-time Polymerase Chain Reaction (RT-PCR Assay)

The real-time RT-PCR assay was performed using was performed using SuperScript™ III Platinum® One-Step Quantitative RT-PCR System Master Mix reagents (catalog no. 11732; Invitrogen, USA) in total 25 μl reaction volume containing 10 μl of RNA sample, 100 copies of IC molecule (SEQ ID NO:4), forward/reverse primers (SEQ ID NO:1 and SEQ ID NO:2 respectively) at a final concentration of 0.3 μM and both probes (SEQ ID NO:3 and 5) at 0.1 μM each in a thermal cycler. Thermal cycling was performed by Stratagene Mx3000P (Stratagene, La Jolla, USA) using the following steps: reverse transcription at 55° C. for 30 min and initial denaturation at 95° C. for 2.5 min, followed by denaturation at 95° C. for 17 s and annealing at 65° C. for 31 s and extension at 68° C. for 32 s. The cycle was repeated 48 times. Fluorescence detection was read at the 65° C. step of each cycle to reveal the positive samples.

The adaptive baseline mode was used for cycle threshold (Ct) determination for data analysis for both the target and IC. If necessary, manual adjustment of the cycle threshold was performed to account for background fluorescence. Single determinations, to mimic routine clinical use, were performed. Each sample run included 2 negative controls to exclude contamination.

### Quantification of HIV-1 Viral Load

Synthetic plasmid standard dilutions were employed as calibration curve when the clinical samples were assayed. Duplicates of each plasmid dilution level were used to generate the calibration curve of the copies per μl against the cycle number, Ct value to determine the copies of HIV-1 RNA per reaction. Clinical samples' viral loads were determined with the extrapolation of the generated Ct value against the calibration curve. Final concentration of viral RNA copies per ml was calculated for each sample taking into account the sample volume used from the eluted volume. The determined Ct value of the IC was used to check for PCR inhibition. A sample with a Ct value of the IC delayed by more than 2 cycles compared to that of equivalent ES was considered invalid due to PCR inhibition and repeated.

### Evaluation of the In-House Assay

Preliminary evaluation of the Sing-IH involved experiments to determine the linear dynamic range, analytical sensitivity and precision using ES. The ability to detect clinically relevant subtypes was assessed against the 1st International Reference Panel for HIV-1 RNA Genotypes (National Institute of Biological Standards and Control, Hertfordshire, United Kingdom).

HIV-1 viral loads in the patient samples were determined by the Abbott RealTime HIV-1 assay (Abbott Molecular Inc, Des Plaines, USA) and the COBAS Taqman HIV-1 test (Roche Molecular Systems, Inc., Branchburg, N.J., USA) in two different laboratories in Singapore using manufacturer's protocol. The quantification ranges provided by the manufacturers of the commercial assays were: Abbott RealTime HIV-1 (40 to 107 copies/ml) and the COBAS TaqMan HIV-1 (48 to 107 copies/ml).

### Statistical Analysis

Microsoft™ Excel 2000 (Microsoft Corporation, USA) and Stata/IC 11.0 (StataCorpLP, College Station, USA) were used for statistical analysis. For clinical samples, comparative histograms were used to assess distribution of log₁₀-transformed viral loads observed for Sing-IH compared to the Abbott RealTime HIV-1 and COBAS TaqMan HIV-1 assays. Bland-Altman plots were used to determine the agreement of the IH-assay with the commercial kits. The Bland-Altman analysis is a measure of the agreement between two instruments measuring on a continuous scale. In brief, differences between the log₁₀-transformed values of the data pairs were graphically represented on the vertical axis against the mean of the log₁₀-transformed values on the horizontal axis. The mean of the log₁₀ paired difference, reflecting the bias, and limits of agreement (mean±2 standard deviation) were plotted on the figures. Probit regression analysis was used to determine the theoretical lower limit of quantitation.

### Results

### Linearity of Dynamic Range

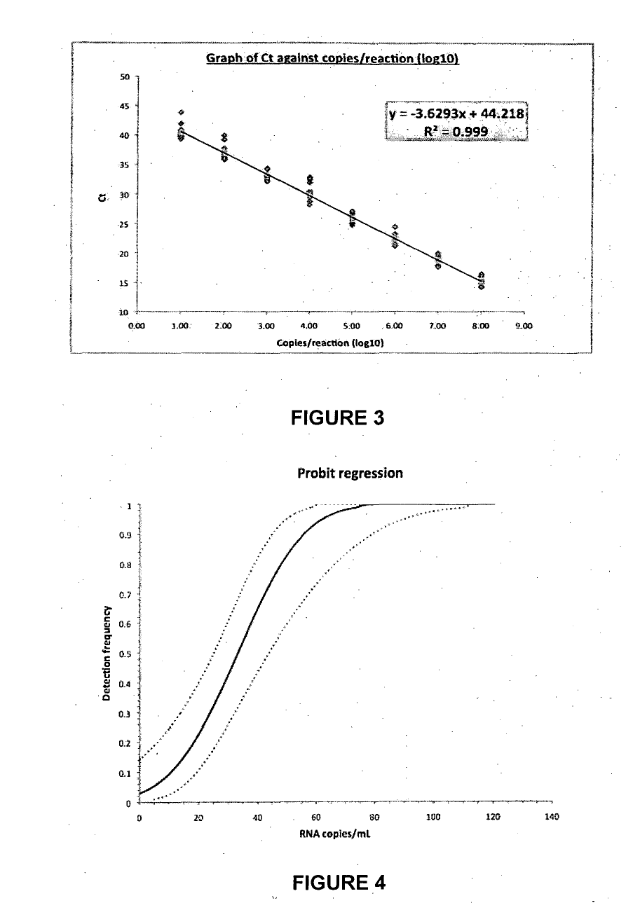

Linearity of the dynamic range of the in-house assay (Sing-IH) was established based on the mean value of the Ct values that were determine from 6 replicate assays per dilution level. Each of the six assays was applied to eight 10-fold serially diluted ES plasmid from three different lots used as targets. These values were plotted against their initial concentration of the ES plasmid in a log scale. Coefficient (r²) of the linear regression of the dynamic range from 10 copies/μl to 10⁸ copies/4 was r²=0.999 is shown in FIG. 3. This indicates that the assay is linear over its quantitation range. For this dynamic range of quantitation, the RT-PCR efficiency is as high as 95.6%.

### Analytical Sensitivity

Analytical sensitivity of the in-house assay was established using serial dilution of plasmid standard (ES plasmid) from 1.6×10⁴ copies/ml to 5 copies/ml which were equivalent to WHO HIV-1 RNA 2^{nd} International Standard 40 000, 12 500, 4 000, 1 250, 400, 125, 40 and 12 copies/ml respectively. The diluted ES plasmids were tested in 4 replicates within a run, with 4 individual runs being carried out. Probit regression was used to determine the projected response rate according to a dose response model. As seen in FIG. 4, at a concentration of 61.5 copies/ml, detection probability was 95% or higher (95% confidence interval, 49.5-89.5 copies/r111). The detection limit of the in-house is 100 copies/ml with 100% detection probability.

### Precision

Intra-assay accuracy was assessed with 8 replicate results for the synthetic plasmid standards containing known 7 log₁₀ copies/μl tested in a single batched experiment. The mean of the calculated viral load was 6.92 log₁₀ copies/4, with CV of 0.825% and SD of 0.04 log₁₀. To ensure that the in-house assay was able to accurately quantify viral load close to the detection limit, plasmid standard is diluted to 1.0 log₁₀ copies/4 and assayed in 8 replicates in single experiment seating. The mean of the measured valued was 0.5 log₁₀ copies/4, with CV of 2.2% and SD of 0.23 log 10.

Inter-assay reproducibility was obtained employing six different experiments, indicating CV % of Ct value<4.2% for all the standard plasmid scalar dilution from 10 copies/reaction to 10⁸ copies/reaction as shown in Table 1.

TABLE 1
Inter-assay variability of the in-house
assay using external plasmid standards.

| Reference standard dilution | | | |
|---|---|---|---|
| (copies/reaction) | Ct Mean values | SD | CV % |
| 10⁸ | 14.54 | 0.47 | 3.21% |
| 10⁷ | 18.16 | 0.76 | 4.19% |
| 10⁶ | 21.64 | 0.70 | 3.25% |
| 10⁵ | 25.26 | 0.46 | 1.83% |
| 10⁴ | 28.84 | 0.90 | 3.14% |
| 10³ | 32.70 | 0.59 | 1.80% |
| 10² | 36.27 | 0.63 | 1.74% |
| 10¹ | 39.99 | 0.90 | 2.26% |

Specificity (Detection of HIV-1 Groups and/or Subtypes)

The ability to detect a range of HIV-1 subtypes was assessed by qualitatively assaying the 1st International Reference Panel for HIV-1 RNA Genotypes (National Institute of Biological Standards and Control, Hertfordshire, United Kingdom). Among the genotypes in the NCBIS genotype panel, the in-house assay was able to detect all of the genotype of group M (A, B, C, D, AE, F, G, AA-GH), and group N with Ct readings between 33.6 to 38.5. Group O was not detected from the panel. As this panel was not a quantitative standard, quantitative comparisons were not performed.

Evaluation of In-House Assay with 329 Patient Samples

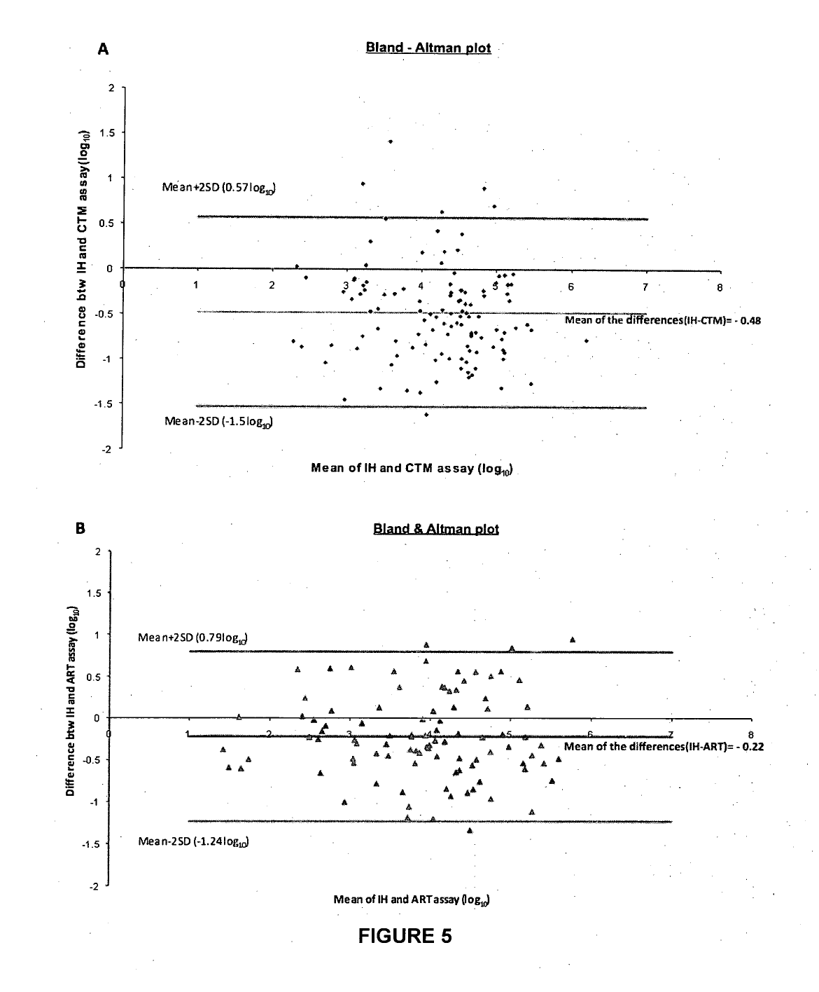

A random group of 329 HIV-1 positive patients' plasma was used to compare the performance of the in-house assay against the commercial kits, ART and CTM. Using a cut off quantifiable level of 50 copies/ml for the commercial and in-house assay, 215 samples were quantified with viral load>50 copies/ml by both the in-house and commercial assays (ART: n=97; CTM: n=118). Agreement between the assays was assessed using a Bland-Altman model (Bland, 1999) as shown in FIG. 5. The mean difference between of the log₁₀ viral load by the in-house and CTM; in-house and ART were −0.48 (limits of −1.61 to 1.41) and −0.22 (limits of −1.34 to 0.95) respectively.

According to the Bland-Altman analysis, among the log₁₀ paired differences, 61% (in-house/ART) and 43% (in-house/CTM) were <absolute 0.5 log₁₀; 33% (in-house/ART) and 42% (in-house/CTM) were within the interval of absolute 0.5 to 1.0 log₁₀; 6% (in-house/ART) and 15% (in-house/CTM) were >1.0 log₁₀. The discrepancy between the in-house/CTM and in-house/ART is not surprising as previous published studies have shown that the mean differences between ART and CTM ranges from −0.24 to 0.51 (Braun, 2007; Foulongne, 2006; Gueudin, 2007). In addition, no funnelling effect was observed in both of the Bland-Altman plots. This showed that the in-house assay correlates better with the Abbott Real time HIV-1 test compared to COBAS TaqMan HIV-1. This is further seen from the linear regression analysis of in-house/ART and in-house/CTM viral load quantitation where the r-values were 0.87 and 0.79 respectively.

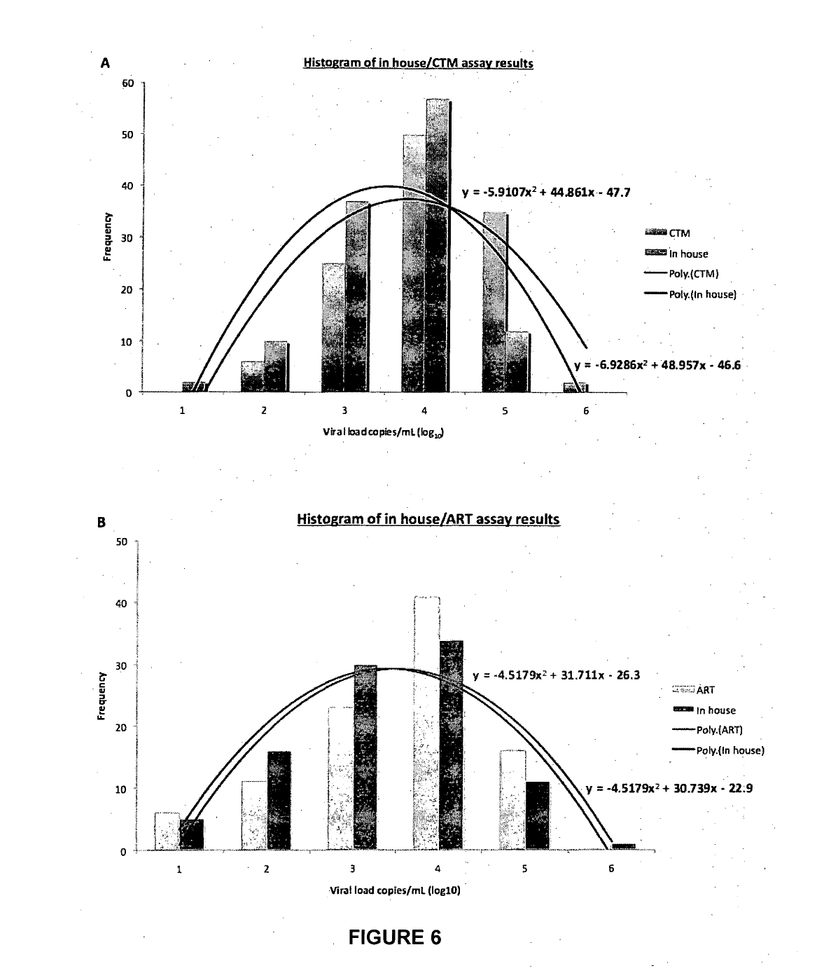

Using the frequency distribution, there were some differences between the in-house and CTM viral load reading distribution. It was observed that there was a shift in the modal peak between in-house and CTM viral load readings from 3.53 to 3.79 log₁₀. Discrepancy was observed at viral reading in the 10⁵ copies/mL, where CTM showed more samples with such higher viral load reading as compared is the in-house's quantitation where the samples are distributed in the 10³-10⁴ copies/ml range. On the other hand, FIG. 6b demonstrated a similarity in the frequency distribution of the HIV-1 viral load values obtained by the in-house and ART assay. The two assays had similar modal peaks at 3.4 log₁₀.

### Sensitivity and Specificity at Clinically Relevant 200 Copies/ml Cut-Off

The performance of the in-house assay was evaluated against the Abbott and Roche assays at the clinical cut-off of 200 copies/ml in 178 and 151 patient samples respectively. Against the Abbott assay as the standard, the in-house assay had a sensitivity of 96.8% and a specificity of 96.4%. With the Roche as standard, the sensitivity was 99.1% and specificity 100%. There were 3 out of 95 positive samples above 200 copies/ml by the Abbott assay which were reported as below 200 copies/mL by the in-house assay (71, 97 and 186 copies/ml). Only 1 sample of the 118 samples reported had more than 200 copies/ml by the Roche assay was reported below 200 copies/ml by the in-house assay (191 copies/ml).

### Lower Cost for In-House HIV-1 Viral Load Assay Test.

HIV patients need to have their HIV-1 viral load test done approximately every 3 months. The in-house assay had the major benefit of lowering the financial burden of the HIV patients. Currently, commercial HIV-1 kit testing in Singapore hospitals costs about S$200 (USD134)/test. Table 2 shows the breakdown of the reagent and consumable costs of the in-house HIV-1 viral load assay to run a patient sample. With the full license and laboratory fees, cost of the in-house assay run will be approximate S$20 (i.e. USD13). This will definitely provide a cheaper option test for the patients.

TABLE 2
Breakdown of the in-house HIV-1 viral load assay costing.

| | Reagent cost | Consumable cost | Subtotal |
|---|---|---|---|
| Sample preparation/sample | S$6.06 | S$1.15 | S$7.21 |
| Real time PCR/sample | S$2.67 | S$7.71 | S$10.38 |
| (Inclusive of 18 plasmid | | | |
| standards) | | | |
| Total cost | | | S$17.59 |

The in-house assays demonstrated consistent results over a log 1 to 8 range per reaction, with a good lower limit of detection by Probit analysis, minimal inter-run variation and quantification of all subtypes except group O, a limitation of the Roche COBAS Amplicor (Ver 1.5) as well (Drosten C et al., 2006). The Blant-Altman analysis also showed comparable results with the 2 SD limits being about 1 log from baseline. The in-house assay was accurate at the clinically relevant cut-off of 200 copies/ml. The minority of samples showing discordance at this cut-off were all detected by the in-house albeit at lower values.

### Example 2

Experiments as substantially explained in Example 1 were repeated in Example 2 and the results provided.

### Analytical Sensitivity

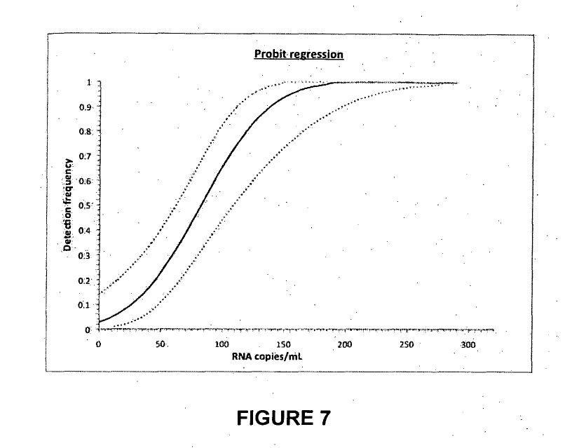

Analytical sensitivity of the in-house assay was established using serial dilution of plasmid standard (ES plasmid) from 1.6×10⁴ copies/ml to 5 copies/ml which were equivalent to WHO HIV-1 RNA 2^{nd} International Standard 40 000, 12 500, 4 000, 1 250, 400, 125, 40 and 12 copies/ml respectively. The diluted ES plasmids were tested in 4 replicates within a run, with 4 runs per dilution. The Probit regression analysis is shown below. As seen in FIG. 7, at a concentration of 154 copies/ml, detection probability was 95% or higher (95% confidence interval, 123.3-223.3 copies/ml). The detection limit of the Sing-IH is 200 copies/ml with 100% detection probability.

Evaluation of the In-House Assay with 329 Patient Samples

Plasma samples from 329 HIV-1 patients on active follow-up were evaluated in comparison with the COBAS TaqMan HIV-1 and Abbott RealTime HIV-1 assays. Using a cut-off quantifiable level of 50 copies/ml for the commercial and in house assay, 227 samples were quantified with viral load>50 copies/ml by both the in house and commercial assays (COBAS TaqMan HIV-1: n=119; Abbott RealTime HIV-1: n=108)).

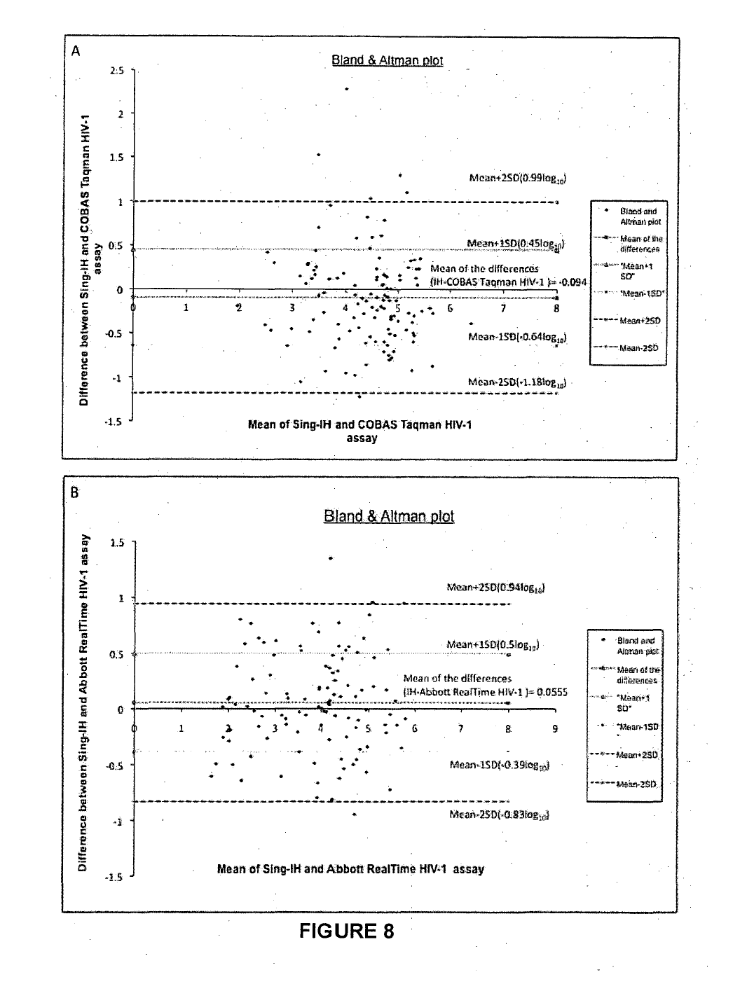

A Bland-Altman analysis was performed (FIGS. 8A and 8B). The mean difference between log₁₀-transformed values of the Sing-IH and COBAS Taqman HIV-1 was −0.094 (95% of values between −1.18 to 0.99). Corresponding values for the Sing-IH and Abbott RealTime HIV-1 were 0.056 (95% of values between −0.83 to 0.94). According to the Bland-Altman analysis, among the log 10-paired differences, 69% of values in both comparisons were within 0.5 log 10.25% of the Sing-IH/COBAS TaqMan HIV-1 values and 30% of the Sing-IH/Abbott RealTime HIV-1 values were within the interval of absolute 0.5 to 1.0 log₁₀; 6% of the Sing-IH/COBAS TaqMan HIV-1 and 1% of the Sing-IH/Abbott RealTime HIV-1 showed greater than absolute 1.0 log 10 difference. No funneling effect was observed in both of the Bland-Altman plots. The linear regression values of the Sing-IH/COBAS TaqMan HIV-1 and the Sing-IH/Abbott RealTime HIV-1 comparison were 0.79 and 0.90 respectively. Linear regression analysis of log₁₀ viral load difference against average log 10 values should no significant correlation. For the Sing-IH/COBAS Taqman HIV-1 comparison, 95% confidence interval of slope −0.03 to 0.17 and for the Sing-IH/Abbott RealTime HIV-1 95% confidence interval of slope −0.06 to 0.11.

The mean difference was evenly distributed across the range of average values, thereby supporting no bias in differences between high versus low quantification values. Regression analysis revealed no statistically significant correlation between differences in log₁₀-transformed values with average log₁₀ values in the Bland-Altman plot.

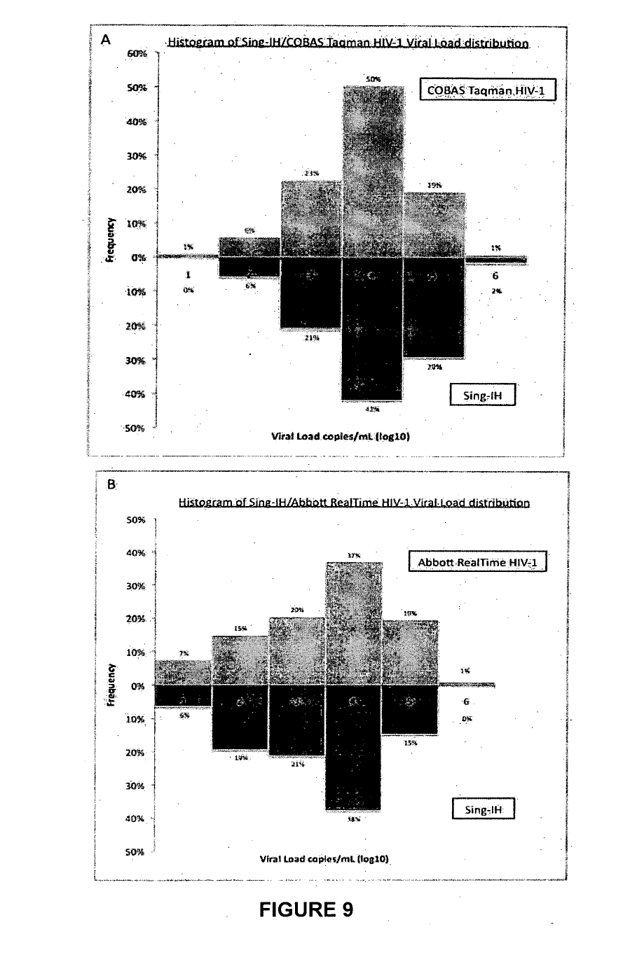

Frequency distribution of the Sing-IH, COBAS TaqMan HIV-1 and Abbott RealTime HIV-1 viral load readings reflected similar unimodal distribution in pair-wise comparison (FIGS. 9A and 9B). For the 119 samples comparing Sing-IH and COBAS TaqMan HIV-1 assay, the modal peaks were 3.68 and 3.79 log₁₀ respectively; for the 108 samples comparing Sing-IH and Abbott RealTime HIV-1 assay, the modal peaks were 3.49 and 3.40 log₁₀ respectively.

### Sensitivity and Specificity at Clinically Relevant 200 Copies/m Cut-Off

To evaluate clinical sensitivity, the performance of the IH-assay was evaluated against the COBAS TaqMan HIV-1 and the Abbott RealTime HIV-1 assays at a cut-off of 200 copies/ml.

The lower limit of the Sing-IH of 200 copies/mL was selected for analysis of clinical sensitivity and specificity. The analysis assumed the commercially available assays as “gold standards” although it is well known in the art that there was no “perfect” HIV-1 RNA viral load assay in view of the extensive genetic.

Against the COBAS TaqMan HIV-1, the sensitivity was 99.1% and specificity 100%. Only 1 sample of the 118 samples reported as more than 200 copies/ml by the Roche assay was reported below 200 copies/ml by the in-house assay.

Against the Abbott RealTime HIV-1 assay as the standard, the IH-assay had a sensitivity of 96.8% and a specificity of 96.4%. 92 samples were reported by both assays with viral load more than 200c/mL. 4 samples were reported positive at this cutoff by the in-house but negative by the Abbott RealTime HIV-1 and 3 samples negative by in-house but positive by Abbott RealTime HIV-1. Assay inhibition was detected in only 1 clinical sample and the repeat result was used in this analysis.

The mean difference of the log₁₀-transformed viral load comparing Sing-IH and commercial assays was less than 0.5 log₁₀. Overall, 96% of samples were within 1 log₁₀ difference between the Sing-IH and competitor assays. At the clinically relevant cut-off of 200 copies/ml, the Sing-IH had excellent agreement of kappa of 0.95 with the commercial assays.

### REFERENCES

1. Bland, J. M., and D. G. Altman. 1999. Measuring agreement in method comparison studies. Stat. Methods Med. Res. 8:135-160.
2. Braun, P., R. Ehret, F. Wiesmann, F. Zabbai, M. Knickmann, R. Kuhn, S. Thamm, G. Warnat, and H. Knechten. 2007. Comparison of four commercial quantitative HIV-1 assays for viral load monitoring in clinical daily routine. Clin. Chem. Lab. Med. 45:93-99.
3. Daar E S et al. Diagnosis of primary HIV-1 infection. Ann Intern Med. 2001 Jan. 2; 134(1):25-9.
4. Drosten C., Panning M, Drexler J F, Hansel F, Pedroso C, Yeats J, de Souza Luna L K, Samuel M, Liedigk B, Lippert U, Stürmer M, Doerr H W, Brites C, Preiser W. Ultrasensitive monitoring of HIV-1 viral load by a low-cost real-time reverse transcription-PCR assay with internal control for the 5′ long terminal repeat domain. Clin Chem. 2006 July; 52(7):1258-66).
5. Foulongne, V., B. Montes, M. N. Didelot-Rousseau, and M. Segond. 2006. Comparison of the LCx human immunodeficiency virus (HIV) RNA quantitative, RealTime HIV, and COBAS AmpliPrep-COBAS TaqMan assays for quantitation of HIV type 1 RNA in plasma. J. Clin. Microbiol. 44:2963-2966.
6. Gueudin, M., J. C. Plantier, V. Lemee, M. P. Schmitt, L. Chartier, T. Bourlet, A. Ruffault, F. Damond, M. Vray, and F. Simon. 2007. Evaluation of the Roche Gobas TaqMan and Abbott RealTime extraction-quantification systems for HIV-1 subtypes. J. Acquir. Immune Defic. Syndr. 44:500-505.

7. Owens D K et al. A Meta-analytic Evaluation of the Polymerase Chain Reaction for the Diagnosis of HIV Infection in Infants. JAMA. 1996 May 1; 275(17)1342-1348.
8. Rekhviashvili N, Stevens W, Marinda E, Gonin R, Stevens G, McIntyre J, Wood R. Clinical performance of an in-house real-time RT-PCR assay using a fluorogenic LUX primer for quantitation of human immunodeficiency virus type-1 (HIV-1). J Virol Methods. 2007 December; 146 (1-2):14-21).
9. Schutten, M., D. Peters, N. K. Back, M. Beld, K. Beuselinck, V. Foulongne, A. M. Geretti, L. Pandiani, C. Tiemann, and H. G. Niesters. 2007. Multicenter evaluation of the new Abbott RealTime assays for quantitative detection of human immunodeficiency virus type 1 and hepatitis C virus RNA. J. Clin. Microbiol. 45:1712-1717.
10. http://www.who.int/mdq/en/.
11. HIV Sequence Compendium 2008 Kuiken C, et al. Eds. Published by Theoretical Biology and Biophysics Group, Los Alamos National Laboratory, NM, LA-UR 06-0680.
12. Johnson, V. A. et al. Top HIV Med. 2009; 17(5): 138-145.

1-4. (canceled)

5. A pair of oligonucleotides comprising at least one forward primer and at least one reverse primer, wherein the forward primer comprises SEQ ID NO: 1 and the reverse primer comprises SEQ ID NO:2.

6. A pair of oligonucleotides comprising at least one forward primer and at least one reverse primer, wherein the forward primer consists of SEQ ID NO: 1 and the reverse primer consists of SEQ ID NO:2.

7. A set of oligonucleotides comprising a pair of oligonucleotides according to claim 5 and at least one probe.

8. The set of oligonucleotides according to claim 7, wherein the probe comprises the nucleotide sequence of SEQ ID NO:3.

9. An amplicon amplified from human immunodeficiency virus type 1 (HIV-1) using at least one forward primer comprising the nucleotide sequence of SEQ ID NO:1 and at least one reverse primer comprising the nucleotide sequence of SEQ ID NO:2.

10. The amplicon according to claim 9, wherein at least one probe comprising the nucleotide sequence of SEQ ID NO:3 is capable of binding to the amplicon.

11. A method of detecting and/or quantitating the presence of human immunodeficiency virus type 1 (HIV-1) in a biological sample, the method comprising the steps of:
(a) providing at least one biological sample;
(b) contacting a pair of oligonucleotides according to claim 5, with at least one nucleic acid in the biological sample, and/or with at least one nucleic acid extracted, purified and/or amplified from the biological sample; and
(c) detecting and/or quantitating any binding resulting from the contacting in step (b) whereby the virus is present when binding is detected.

12. A method of detecting the presence of human immunodeficiency virus type 1 (HIV-1) in a biological sample, the method comprising the steps of:
(a) providing at least one biological sample;
(b) contacting a set of oligonucleotides according to claim 7, with at least one nucleic acid in the biological sample, and/or with at least one nucleic acid extracted, purified and/or amplified from the biological sample; and
(c) detecting any binding resulting from the contacting in step (b) whereby the virus is present when binding is detected.

13. The method according to claim 11, further comprising the step of mixing an internal molecule (IC) and a probe specific to the IC with the biological sample after step (a).

14. The method according to claim 13, wherein the IC comprises the nucleotide sequence of SEQ ID NO:4 and the probe specific to the IC comprises the nucleotide sequence of SEQ ID NO:5.

15. A method of amplifying human immunodeficiency virus type 1 (HIV-1) nucleic acid, wherein said method comprises carrying out a polymerase chain reaction using at least one forward primer comprising the nucleotide sequence of SEQ ID NO:1 and at least one reverse primer comprising the nucleotide sequence of SEQ ID NO:2.

16. The method according to claim 15, wherein the method further comprises using at least one probe comprising the nucleic acid of SEQ ID NO:3.

17. (canceled)

18. A kit for the detection of human immunodeficiency virus type 1 (HIV-1), the kit comprising at least one pair of oligonucleotides according to claim 5.

19. A kit for the detection of human immunodeficiency virus type 1 (HIV-1), the kit comprising at least one set of oligonucleotides according to claim 7.

20. The pair of oligonucleotides according to claim 5, wherein the oligonucleotides are capable of binding to and/or being amplified from human immunodeficiency virus type 1 (HIV-1).

21. The set of oligonucleotides according to claim 7, wherein the oligonucleotides are capable of binding to and/or being amplified from human immunodeficiency virus type 1 (HIV-1).
